(19) United States
(12) Patent Application Publication
(10) Pub. No.: US 2018/0021425 A1
(43) Pub. Date: Jan. 25, 2018

(54) CYTOMEGALOVIRUS-BASED VACCINE EXPRESSING EBOLA VIRUS GLYCOPROTEIN

(71) Applicants: UNIVERSITY OF PLYMOUTH, Plymouth Devon (GB); THE REGENTS OF THE UNIVERSITY OF CALIFORNIA, Oakland, CA (US)

(72) Inventors: Michael Arthur JARVIS, Totnes Devon (GB); Aisling Aoife MURPHY, Plymouth Devon (GB); Heinrich FELDMANN, Hamilton, MT (US); Andrea Marzi, Hamilton, MT (US); Ilhem Messaoudi Powers, Riverside, CA (US)

(21) Appl. No.: 15/547,365

(22) PCT Filed: Jan. 28, 2016
(86) PCT No.: PCT/EP2016/051854

§ 371 Date: Jul. 28, 2017

(30) Foreign Application Priority Data
Jan. 30, 2015 (GB) 1501523.3

(51) Int. Cl. A61K 39/12; C12N 7/00; A61K 39/02

(52) U.S. Cl. CPC A61K 39/12; A61K 39/02; C12N 7/00; C12N 2710/16143; A61K 2039/5256

(57) ABSTRACT

A recombinant herpesvirus-based vector comprising a nucleic acid sequence encoding a heterologous antigen and a promoter for controlling the expression of the antigen, in which the promoter is expressed at a time selected to provide a required immune response in a subject.

### CROSS-REFERENCE TO RELATED APPLICATIONS

This application is a national stage entry under 35 U.S.C. 371 of International Application PCT/EP2016/051854 filed Jan. 28, 2016 (currently pending). International Application PCT/EP2016/051854 cites the priority of British Patent Application 1501523.3, filed on Jan. 30, 2015.

### BACKGROUND

Herpesviridae is a large family of DNA viruses that cause diseases in animals, including humans. The members of this family are also known as herpesviruses.

All herpesviruses are composed of relatively large double-stranded, linear DNA genomes encoding 100-200 genes encased within an icosahedral protein cage called the capsid which is itself wrapped in a protein layer called the tegument containing both viral proteins and viral mRNAs and a lipid bilayer membrane called the envelope. This whole particle is known as a virion.

Cytomegalovirus (CMV)-based vaccines, as well as other herpesvirus-based vaccines, are on the horizon as a promising addition to our arsenal against infectious disease and cancer. These herpesvirus-based vectors are unique, not only in the high level of T cell immunity they induce against their heterologous encoded pathogen (or cancer) target antigen, but also in the durability of the immunity and in its ‘immediate-effector’ quality.

CMV is a member of the beta subclass of the herpesvirus family. To date, CMV is the best characterized of the herpesvirus-based vaccine vectors.

A RhCMV-based vaccine against the monkey version of HIV (simian immunodeficiency virus, SIV) was recently shown to induce protection against systemic infection in rhesus macaques—a level of protection never observed before for any SIV vaccination regimen.

CMV-based vaccines have also recently been shown to protect against such diverse pathogens and diseases as Ebola virus, tetanus and prostate cancer in mouse model systems using mouse CMV (MCMV).

Although CMV-based (and other less developed herpesvirus-based) vaccines have been shown to induce substantial levels of T cell responses against the encoded heterologous target antigens, the induction of substantial antibody responses against these targets has not been achieved by these vectors. This T cell bias is regarded as a limitation of these vaccines for their broad application to target infectious diseases and cancer. A number of references (patents and publications) are supplied as evidence supporting the generalized inability of herpesvirus-based vaccines to induce antibody responses.

### BRIEF SUMMARY OF THE INVENTION

The present invention relates generally to immune responses and more specifically to promoter usage providing differential immune response modulation.

All documents cited or referenced herein and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The present invention is derived from unexpected findings from our Ebola virus challenge study in rhesus macaques vaccinated with a rhesus CMV expressing an Ebola virus target antigen (Ebola virus glycoprotein, GP).

A characteristic of herpesvirus genes is that they differ in their kinetics of expression in relationship to the herpesvirus replication cycle. Based on the time of expression, herpesvirus genes are classified into immediate-early (IE), early (E), or late (L) genes.

In all the RhCMV-based vectors made prior to our Ebola study, the heterologous target antigen has been place under a heterologous promoter that was expressed either at IE/E times or that has remained uncharacterized (this was also the case for other non-human primate herpesvirus-based vectors and MCMV). In contrast, the CMV vector used in our Ebola study was designed to place the Ebola virus GP under the control of the RhCMV endogenous pp65b promoter by replacement of the non-essential Rh112 (human CMV UL83 orthologue) gene with the Ebola virus GP gene. This places GP under control of the pp65 promoter which is expressed at L times. The use of this L promoter resulted in a complete reversal of the normal immune response associated with CMV and other herpesvirus-based vectors in the primate model. Specifically, it resulted in the immune response induced by the rhesus CMV vaccine being heavily biased towards induction of GP-specific antibodies, with minimal induction of GP-specific T cell responses.

This is a completely unexpected finding to those in the herpesvirus vaccine field based on the current understanding of these vaccines. Our idea concept, which has been reduced to practice in our recent study, is therefore that by utilizing promoters that express with different kinetics in herpesvirus-based vaccines we can now create vaccines that will bias the target pathogen-specific immune response towards either antibody production with diminished T cell responses (by use of a promoter expressed at L times), or towards T cell responses with diminished antibody responses (by use of a promoter expressed at IE and E times).

By use of promoters that are intermediate in their kinetics of expression (E-L), we would reasonably assume that a balanced antibody and T cell response would be achieved. Alternatively a mixture of vaccines using different kinetic classes of promoters to drive target antigen expression, either within distinct vaccines or within the same vaccine construct, would enable a balanced antibody and T cell response to be achieved. In this fashion, it will be possible to create vaccines that induce the desired balance of antibody and T cell responses against any target pathogen or cancer antigen.

The present invention is based on the principle of promoter usage to provide differential immune response modulation.

According to an aspect of the present invention there is provided a method of preparing a recombinant herpesvirus-based vector comprising the steps of: providing a nucleic acid sequence encoding a heterologous antigen; selecting a promoter for controlling the expression of the antigen, in which the promoter is selected to express at a time selected to provide a required immune response in a subject.

The present invention can be used to drive differential antibody and T cell response in a subject Selection of a temporal promoter can be used to drive a response in one direction or another, or to provide a balanced cell-mediated response.

According to a further the present invention there is provided a recombinant herpesvirus-based vector comprising a nucleic acid sequence encoding a heterologous antigen and a promoter for controlling the expression of the antigen, in which the promoter is expressed at a time selected to provide a required immune response in a subject.

The present invention may comprise a fully replication competent vector and/or an attenuated vector and/or a replication defective vector and/or any of the above vectors deleted or modified for immunomodulator genes.

The promoter may be selected to provide an immune response in a subject which is biased towards an antibody response in a subject.

The promoter may be selected to provide an immune response in a subject which is biased towards a T cell response in a subject.

The promoter may be expressed at E, IE or L times.

The herpesvirus-based vector may be selected from: Iltovirus, Proboscivirus, Cytomegalovirus, Mardivirus, Rhadinovirus, Macavirus, Roseolovirus, Simplexvirus, Scutavirus, Varicellovirus, Percavirus, Lymphocryptovirus, Muromegalovirus.

The herpesvirus-based vector may be a CMV-based vector.

The CMV-based vector may be selected from the non-exclusive list of human CMV, rhesus CMV, simian CMV, chimpanzee CMV, murine CMV and gorilla CMV.

The present invention also provides a recombinant herpesvirus-based vector comprising a nucleic acid sequence encoding a heterologous antigen and a promoter for controlling the expression of the antigen, in which the promoter is expressed at L times.

The present invention also provides a recombinant CMV-based vector comprising a nucleic acid sequence encoding a heterologous antigen and a promoter for controlling the expression of the antigen, in which the promoter is an endogenous CMV promoter that is expressed at L times, or a heterologous promoter expressed with comparable kinetics.

The present invention also provides a CMV-based vaccine with enhanced antibody production, comprising a recombinant CMV-based vector comprising a nucleic acid sequence encoding a heterologous antigen and a promoter for controlling the expression of the antigen, in which the promoter is expressed at L times.

The present invention also provides a vaccine comprising a recombinant herpesvirus-based vector, the vector construct comprising at least two nucleic acid sequences encoding heterologous antigens and each being under the control of a promoter, in which the promoter for each sequence is selected from a different kinetic class such that a predetermined balanced immune response can be achieved in a subject.

The present invention also provides a vaccine comprising a mixture of two or more types of recombinant herpesvirus-based vector, the vector types each comprising a nucleic acid sequence encoding a heterologous antigen and a promoter for controlling the expression of the antigen, in which the promoter in each type of vector is selected from a different kinetic class such that a predetermined balanced immune response can be achieved in a subject.

The heterologous antigen provided in aspects and embodiments of the present invention may be a pathogen-specific antigen or a tumor antigen, for example a human pathogen-specific antigen or a tumor antigen

The heterologous antigen may be a pathogen-specific antigen, for example a human pathogen-specific antigen

The pathogen from a (for example human) pathogen may be a viral antigen.

The antigen may be selected from a non-exclusive list of the following: human immunodeficiency virus, simian immuno-deficiency virus, Kaposi's sarcoma-associated herpesvirus, herpes simplex virus 1, herpes simplex virus 2, herpes virus B, Epstein Barr virus, hepatitis B virus, human papillomavirus, influenza virus, monkeypox virus, West Nile virus, Chikungunya virus, Ebola virus, hepatitis C virus, poliovirus, dengue virus, herpes virus B, Marburg virus, SARS virus, MERS virus.

In some aspects and embodiments a viral protein, an epitope or antigenic fragment thereof may be used as a heterologous antigen.

In some aspects and embodiments the pathogen-specific antigen may be a bacterial antigen.

In some aspects and embodiments the pathogen-specific antigen may be a fungal antigen.

In some aspects and embodiments the pathogen-specific antigen may be a protozoan antigen.

In some aspects and embodiments the pathogen-specific antigen may be a helminth antigen.

The vector/vaccine of the present invention may comprise, include or consist of one or more nucleic acid sequence as described and defined herein.

Genomes and sequence listings referred to and used in this study are:

### AC146851

### AC146904

### AC146905

### AC146906

### AC146907

### AC146999

### AY186194

### DQ120516

### EF990255

### JN227533

### FJ483968

### U27883

### U27627

### U27469

### U27770

### U27471

### U27238

### AC090446

### AF480884

### AY446894

### JQ795930

The present invention also provides a composition comprising the vaccine or vector as described herein and a pharmaceutically acceptable carrier.

The present invention also provides a method of treating a subject with an infectious disease, or at risk of becoming infected with an infectious disease comprising selecting a subject in need of treatment and administering to the subject the recombinant vector or vaccine described herein or the composition described herein.

The present invention also provides for the use of the JQ795930 RhCMV vector or JQ795930 modified by additional attenuation as a vector for use in the treatment of prevention of disease in humans; for example, the use of this vector in a vaccine for the treatment of ebolavirus.

The present invention also provides a replication deficient HCMV-based vector including a human pp65 promoter controlling expression of ebolavirus glycoprotein. The vector may be derived from a diseased or a non-diseased source. Use of such a vector in the treatment or prevention of ebolavirus in humans is also provided.

The present invention also provides a replication deficient HCMV-based vector including a human EF-1α promoter controlling expression of ebolavirus glycoprotein. The vector may be derived from a diseased or a non-diseased source. Use of such a vector in the treatment or prevention of ebolavirus in humans is also provided.

The present invention also provides a method of treating a subject with a pre-existing infectious disease therapeutically, or at risk of becoming infected with an infectious disease prophylactically comprising selecting a subject in need of treatment and administering to the subject the recombinant vector or vaccine described herein or the composition described herein.

The present invention also provides a method of treating a subject with cancer, or at risk of developing cancer comprising selecting a subject in need of treatment and administering to the subject the recombinant vector or vaccine as described herein or the composition described herein.

The present invention also provides use of a vaccine, vector or composition as described herein for the prevention or treatment of a disease.

The present invention also provides a method of providing a modulated immune response comprising the steps of:
providing a herpesvirus-based vector
providing the vector with a nucleic acid sequence encoding a heterologous antigen
selecting a promoter for controlling expression of the antigen
in which selection of the promoter is determined by the immune response type required.

One aspect of the present invention relates to a CMV-based vaccine with enhanced antibody production provides nonhuman primates protection against ebolavirus.

The two independent 2014 ebolavirus (Zaire) (ZEBOV) outbreaks in West Africa and the Democratic Republic of the Congo (DRC) emphasize the public health problems filoviruses pose due to their lethality, unpredictable emergence, and localization to the poorest areas of the world¹. Wildlife, primarily bats and great apes, play a critical role in filovirus transmission to humans by serving as reservoir or amplification species²⁻⁵. Interaction of humans with these species, either directly or by exposure to their habitat, has frequently been associated with human filovirus outbreaks^{3,6}. ‘Self-disseminating’ cytomegalovirus (CMV)-based filovirus vaccines are one strategy to achieve filovirus-specific immunity in wildlife populations and block transmission to humans. Extending on earlier mouse studies⁷, rhesus CMV (RhCMV) expressing ZEBOV glycoprotein (GP) was shown to provide protective immunity from a highly lethal, low passage (7U) ZEBOV challenge. Surprisingly, the recombinant RhCMV GP-expressing ZEBOV vaccine induced high levels of GP-specific antibodies, but with minimal induction of GP-directed cellular immunity. We hypothesize that this bias towards humoral immunity targeting the heterologous target antigen, something not observed previously for RhCMV-based vectors, may result from novel promoter selection in the recombinant vaccine. In addition to showing substantial promise for development as a ‘self-disseminating’ vaccine to target ebolavirus in wild ape populations, this study identifies a potential strategy by which herpesvirus-based vectors can be rationally designed towards humoral, cellular or both arms of the adaptive immune response based on promoter selection.

The 2014 ZEBOV outbreak in the West African states of Guinea, Sierra Leone and Liberia is a stark example of the devastating effect of filoviruses following establishment of human-to-human transmission in densely populated areas with poor infrastructure¹. Following introduction into the human population, ebolavirus is maintained by human-to-human transmission, and current interventional strategies are aimed at decreasing the reproductive number (R₀) of transmission to R₀<1⁸. In previous outbreaks, this has been achieved by timely implementation of standard public health measures consisting of rapid identification and isolation of infected individuals, contact tracing and use of stringent barrier nursing procedure during patient care⁹. As evidenced in the West African outbreak, such interventional strategies may be insufficient to contain epidemic spread when delayed or under-resourced, especially following movement of ebolavirus into the urban environment of countries with inadequate public health systems. In the current outbreak, R₀ fell from its early epidemic peak estimate of between 1.71 and 2.02, but as of Sep. 14, 2014 the net reproduction number Rₜ remains >1, and the number of cumulative confirmed and probable cases has been predicted to exceed 20,000 by early November¹.

All human ebolavirus outbreaks are believed to result from zoonotic transmission from wild animal reservoir or transmission/amplification species³. Prevention of the initial zoonotic introduction from wildlife into the human population has therefore been proposed as an additional and complementary strategy to control human ebolavirus outbreaks^{3,7,10}. Fruit bats have been identified as one probable reservoir for ebolavirus, and direct contact or exposure to environments inhabited and frequented by bats has been associated with human ebolavirus outbreaks^{3,6,11}. Great apes (chimpanzees and western lowland gorillas) are regarded as a second main source of zoonotic ebolavirus transmission^{3,4,12,13}, and vaccination of African great apes has been proposed as one possible strategy to prevent ebolavirus transmission to humans^{3,7,10}. This approach may be especially suited to heavily under-resourced areas where the healthcare infrastructure is unable to control human-to-human transmission once ebolavirus infection has been established within urban populations. Ebolavirus is also highly lethal in African great apes^{2,4,12,14-16}, and has resulted in a substantial reduction in the world gorilla population. Ebolavirus is therefore regarded as a major threat to the survival of chimpanzees and gorillas in the wild. In response to this threat, western lowland gorillas were upgraded to ‘Critically Endangered’ by the World Conservation Union in 2007¹⁷. Vaccination of great apes is gaining support from primate conservationists due to its potential to stabilize ape populations against the devastating effects of ebolavirus.

Wild apes inhabiting geographically inaccessible tropical rainforests pose significant hurdles to conventional vaccination based on direct inoculation or baiting of individual animals. We recently proposed the use of a cytomegalovirus (CMV)-based ‘self-disseminating’ vaccine as one strategy to achieve the necessary vaccine coverage in these inaccessible and hostile environments⁷. In this scenario, high coverage would occur following animal-to-animal spread of the vaccine from a few initial directly inoculated ‘founder’ vaccines. CMV is a benign, species-specific member of the β-herpesvirus subfamily^{18,19}. Due to its ability to induce substantial durable levels of T cells against heterologous encoded target antigens, CMV has gained considerable interest for development as a vaccine vector platform²⁰⁻²⁴. CMV is also able to spread easily through its host population, even in CMV-seropositive individuals^{7,25-28}. In previous studies, we have shown the ability of a single dose of a murine CMV (MCMV) expressing a CD8 T cell epitope from nucleoprotein (NP) of ZEBOV (MCMV/ZEBOV-NP_{CTL}) to induce durable, ZEBOV-specific CD8⁺ T cell immunity that remained protective against lethal ZEBOV challenge until >14 weeks post-vaccination (the latest time point measured in the studies).

Different aspects and embodiments of the invention may be used separately or together.

Further particular and preferred aspects of the present invention are set out in the accompanying independent and dependent claims. Features of the dependent claims may be combined with the features of the independent claims as appropriate, and in combination other than those explicitly set out in the claims.

The present invention will now be more particularly described, by way of example, with reference to the above accompanying drawings, in which:

### BRIEF DESCRIPTION OF THE DRAWINGS

FIGS. 1A-1C show construction and characterization of RhCMV vectors engineered to express EBOV (Zaire) GP (designated RhCMV/ZEBOV-GP).

FIG. 1A shows a schematic representation of RhCMV/ZEBOV-GP predicted topology. A codon-optimized full-length ZEBOV glycoprotein (GP) (Zaire) was inserted within the RhCMV genome (68.1) to replace the endogenous Rh112 (pp65b). This approach places GP under the control of the endogenous RhCMV Rh112 promoter.

FIG. 1B shows the multi-step growth analysis of RhCMV/ZEBOV-GP. RFs were infected at a MOI of 0.01 with either RhCMV/WT, RhCMV/ZEBOV-GP[2-8] or RhCMV/ZEBOV-GP[6-1]. Supernatant was collected at days indicated post infection and titered in a TCID₅₀ assay. The assay was performed in triplicate and standard deviation is shown.

FIG. 1C shows western analysis of RhCMV/ZEBOV-GP in RFs that exhibit stable expression of ZEBOV GP until at least passage 7. Western blot analysis of RhCMV/ZEBOV-GP infected cell lysates based on V5 epitope tag and anti-GP MAb detection, showed that GP was expressed at high levels, and that expression was stable over multiple passages (passage 7). V5 activity was observed against 3 bands as predicted [110 kDa preGPer (not shown); 160 kDa preGP (not shown) and 26 kDa GP2]. Anti-GP MAb was used to detect GP1 (140 kDa) as the V5 tag is localized at the carboxyl-terminus of preGP.

FIG. 2 shows that RhCMV/ZEBOV-GP expresses ZEBOV-GP at late times of replication. Western analysis RhCMV/ZEBOV-GP in RFs showing ZEBOV GP expression at L times of replication. RFs were infected at a MOI of 0.01 with either RhCMV/WT, RhCMV/ZEBOV-GP[2-8] or RhCMV/ZEBOV-GP[6-1]. Cell lysates were collected at days indicated post infection and analysed by western blot. Accumulation of ZEBOV-GP was compared to accumulation of viral proteins known to be expressed with IE (IE-1) and L (pRh112) kinetics.

FIGS. 3A-3C. show that RhCMV/ZEBOV-GP induces high levels of antibodies against ZEBOV-GP, with absence of ZEBOV-GP directed T cell responses in RMs.

FIG. 3A shows a timeline of ‘vaccine’ and ‘challenge’ phases and sampling schedule.

FIG. 3B shows the time course of CD4⁺ and CD8⁺ T cell responses against IE-1, pRh112 (pp65b) and ZEBOV GP. T cells were analysed by ICS following incubation with overlapping peptide pools in the presence of BFA. Levels of responding cells (TNFα and IFNγ double-positive) in individual RMs are shown at times indicated. T cell responses against endogenous RhCMV antigens (IE-1 and Rh112) were observed in all animals, while no responses against ZEBOV GP were detected at any time.

FIG. 3C shows the time course of antibody responses against ZEBOV GP. Total IgG antibody levels against ZEBOV GP were measured by ELISA at times indicated. Antibodies were detected after the initial RhCMV/ZEBOV-GP vaccination, and then increased further following the boost at day −28. The drop in antibody levels observed at 4 days post-challenge is consistent with antibody consumption during control of ZEBOV infection.

FIG. 4A-4I show clinical parameters in RhCMV/WT and RhCMV/ZEBOV-GP vaccinated animals. Changes in various clinical parameters were measured over the duration of the study: Kaplan-Meier survival curves (FIG. 4A), temperature (FIG. 4B), daily clinical scores (FIG. 4C), viremia (FIG. 4D), WBCs (FIG. 4E), platelets (FIG. 4F), AST levels (FIG. 4G), ALT levels (FIG. 4H) and ALP levels (FIG. 4I).

FIG. 5 shows genomic characterization of RhCMV/ZEBOV-GP BAC. DNA from two independent clones of RhCMV/ZEBOV-GP [2-8 and 6-1] were digested with EcoRI followed by electrophoresis. The comparable digest pattern between RhCMV/ZEBOV-GP [6-1] and RhCMV/WT BAC shows the lack of any gross genomic rearrangement.

FIG. 6 shows original dot-plot data from flow cytometry experiments presented graphically in FIGS. 3A-3C.

Supplemental Table 1. Combined Sanger and NGS Sequencing of BACs and reconstituted RhCMV/ZEBOV-GP (Clone 2-8 and 6-1).

Supplemental Table 2. Total and neutralizing antibody levels pre- and post-ZEBOV challenge.

FIG. 7 shows clinical findings during the ZEBOV challenge phase. Fever was defined as ≧1° C. above baseline. Mild rash was defined as areas of petechiae covering less that 10% of the skin, moderate rash was defined as areas of petechiae covering 10-40% of the skin and severe rash was defined as areas of petechiae covering >40% of the skin. Leukocytopenia and thrombocytopenia were defined as a >30% decrease in numbers of WBCs and platelets, respectively. Leukocytosis and thrombocytosis were defined as a twofold or greater increase in numbers of WBCs and platelets above baseline levels, where WBC count >11,000. Elevated ALT, AST and ALP levels were defined as: ↑ (>2-fold; <4-fold increase), ↑↑ (>4-fold; <5-fold increase) and ↑↑↑ (>5-fold increase).

### DESCRIPTION

Example embodiments are described below in sufficient detail to enable those of ordinary skill in the art to embody and implement the systems and processes herein described. It is important to understand that embodiments can be provided in many alternate forms and should not be construed as limited to the examples set forth herein.

Accordingly, while embodiments can be modified in various ways and take on various alternative forms, specific embodiments thereof are shown in the drawings and described in detail below as examples. There is no intent to limit to the particular forms disclosed. On the contrary, all modifications, equivalents, and alternatives falling within the scope of the appended claims should be included. Elements of the example embodiments are consistently denoted by the same reference numerals throughout the drawings and detailed description where appropriate.

The terminology used herein to describe embodiments is not intended to limit the scope. The articles “a,” “an,” and “the” are singular in that they have a single referent, however the use of the singular form in the present document should not preclude the presence of more than one referent. In other words, elements referred to in the singular can number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof.

Unless otherwise defined, all terms (including technical and scientific terms) used herein are to be interpreted as is customary in the art. It will be further understood that terms in common usage should also be interpreted as is customary in the relevant art and not in an idealized or overly formal sense unless expressly so defined herein.

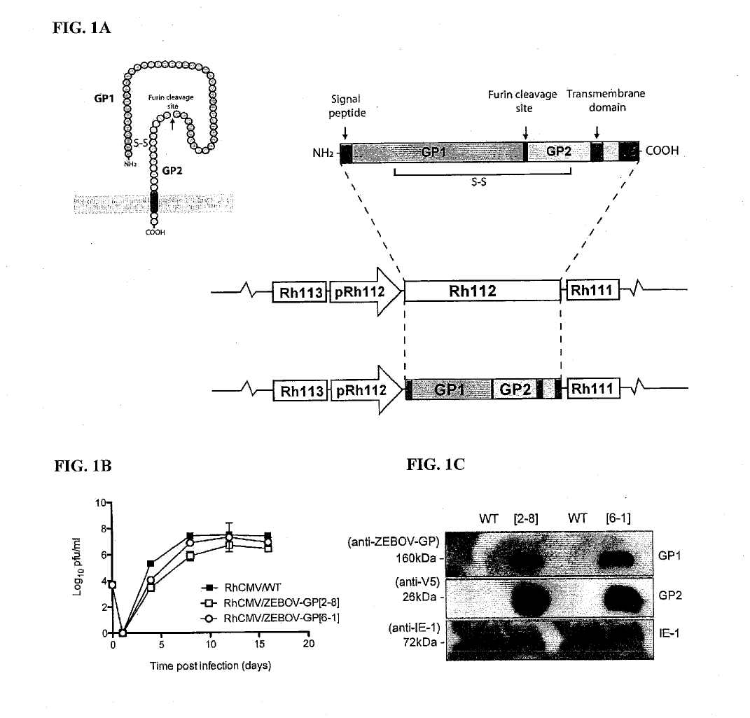

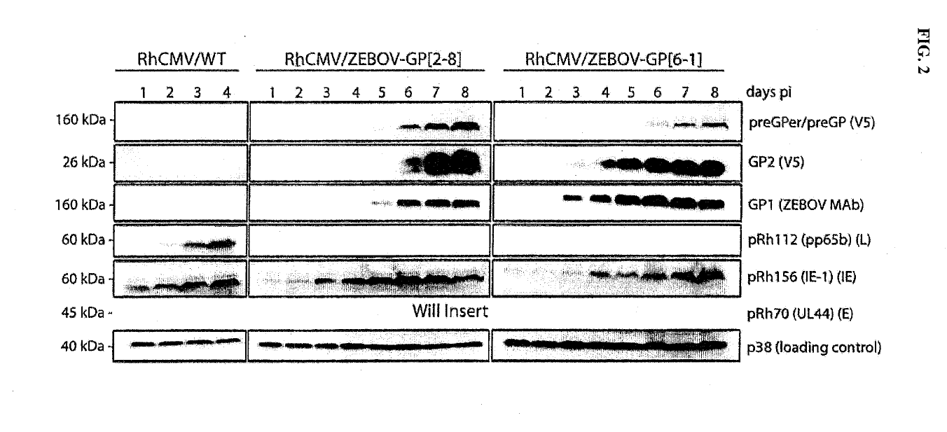

The aim of the current study was to assess the protective efficacy of a RhCMV-based ZEBOV vaccine expressing full length ZEBOV GP (RhCMV/ZEBOV-GP) against a low-passage lethal ZEBOV challenge in the macaque model, which is regarded as the ‘gold standard’ for translation of protection to great apes, including humans²⁹. GP was chosen as the ZEBOV target, as this antigen has been shown to be a target of protective immunity when expressed from other vaccine platforms^{30,31}. All RhCMV/ZEBOV-GP vectors were constructed using bacterial artificial chromosome (BAC)-based technology as previously described^{20,32,33}. A schematic of RhCMV/ZEBOV-GP is shown in FIG. 1A. RhCMV-based vectors used in previous studies have utilized heterologous promoters for target antigen expression^{20,33}. In the RhCMV/ZEBOV-GP vector, expression of the full length codon-optimized GP ZEBOV target gene³⁴ was placed under control of the endogenous RhCMV Rh112 (pUL83b; pp65b) promoter, which is normally expressed at late times of virus replication. Similar to all herpesviruses, genes of CMV differ in their time of expression during the virus replication cycle, and are classified as immediate-early (IE), early (E) or late (L) genes. In addition to being one of the most highly expressed promoters, the endogenous Rh112 L gene promoter was chosen to potentially increase stability of expression of the GP transgene by delaying its expression until later times in the replication cycle. This strategy results in deletion of the RhCMV Rh112 open-reading frame (ORF), which is dispensable for secondary RhCMV persistent infection and induction of RhCMV-directed T cell immunity in CMV-immune rhesus macaques³⁵. GP was also V5 epitope tagged at the carboxyl terminus to facilitate detection. FIG. 1B shows in vitro replication kinetics of two independent RhCMV/ZEBOV-GP clones (2-8 and 6-1) in primary rhesus fibroblasts (RFs). FIG. 1C shows stable GP expression until at least passage 7 in RhCMV/ZEBOV-GP infected RFs using either a V5-specific antibody or a GP-specific monoclonal antibody that binds to a region within the N-terminal region of GP for detection (see FIG. 1A schematic). Time of GP expression was then analyzed to determine whether the heterologous ZEBOV target antigen was still expressed with L kinetics. FIG. 2 shows that GP is expressed at later times of RhCMV replication consistent with its control by the L Rh112 promoter.

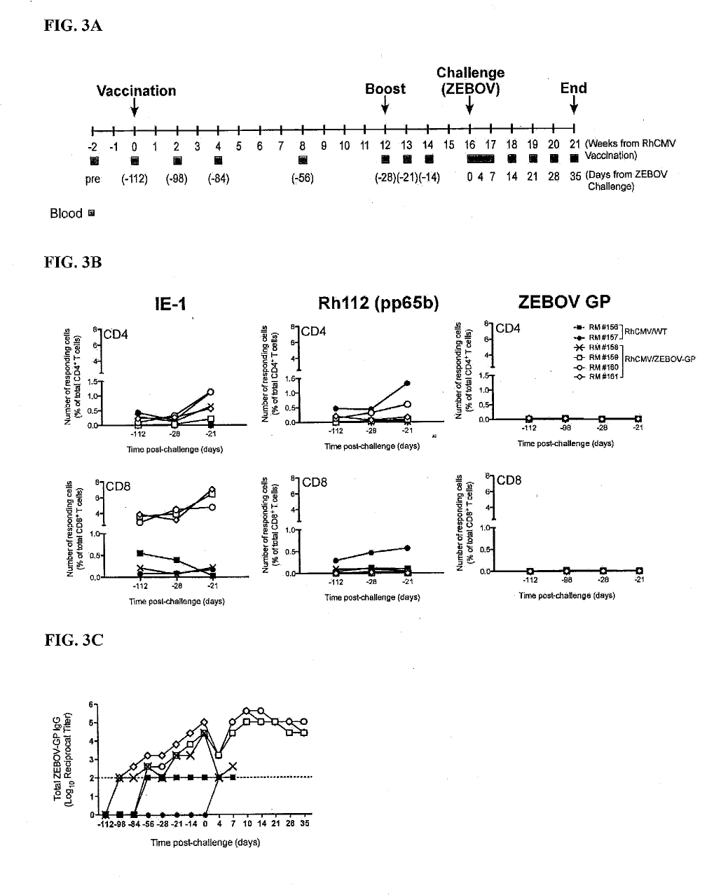

A group of 4 Rhesus Monkeys (RMs) were inoculated with RhCMV/ZEBOV-GP in order to assess vaccine immunogenicity and efficacy (see schematic: FIG. 3A). Two additional animals received the parental 68-1 BAC-derived RhCMV. The ability of CMV to infect the host is not affected by CMV immune status, and all RMs were RhCMV seropositive at the time of inoculation as a consequence of natural RhCMV infection. At day −112, the 4 animals allocated to the vaccine arm were inoculated with an equal mixture of two independently derived RhCMV/ZEBOV-GP clones (2-8 and 6-1) via the subcutaneous (s.c.) route for a total dose of 1×10⁷pfu. The 2 control animals received a comparable s.c. inoculation of parental 68-1 RhCMV (total dose of 1×10⁷pfu). Animals were then boosted in an identical fashion at day −28. RMs were followed immunologically for T cell (FIG. 3B) and antibody responses (FIG. 3C) during the vaccine phase. Previous studies, using RhCMV expressing simian immunodeficiency virus (SIV) and human tuberculosis (TB)-derived antigens under control of heterologous promoters, have shown immune responses against the target antigens to be heavily shifted towards induction of high levels of effector memory (TEM)-biased T cell responses, with only low or undetectable levels of non-neutralizing antibodies^{20,32,33}. We were therefore surprised to observe a reversal of this immunological phenotype in our RMs vaccinated with RhCMV/ZEBOV-GP. In contrast to these earlier studies, only background levels of CD4⁺ or CD8⁺ T cells were present against the GP antigen, even following the ‘boost’ at day −28. Although variable, T cell responses against endogenous RhCMV antigens (IE1 and pp65b) were observed in all animals. RhCMV/ZEBOV-GP vaccination was, however, associated with high levels of antibodies against GP. These GP-specific antibodies were detected following the initial vaccination, and then increased following the day −28 boost (day 0, median: 25600, range: 25600 to 102400). This antibody-biased immune response directed against the heterologous GP antigen is a phenotype that has not been seen previously for any RhCMV-based vaccine³², nor any other recombinant primate herpesvirus-based vector³⁶.

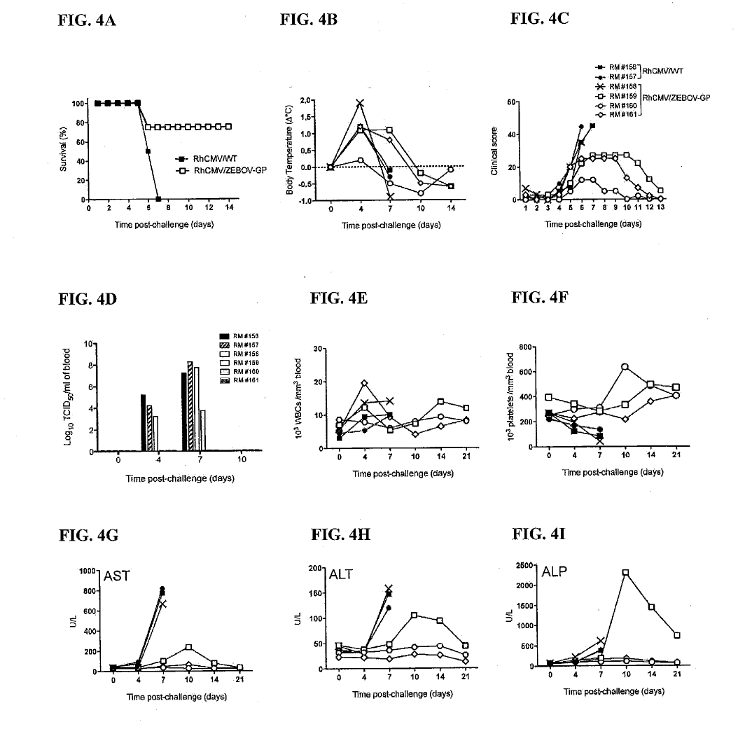

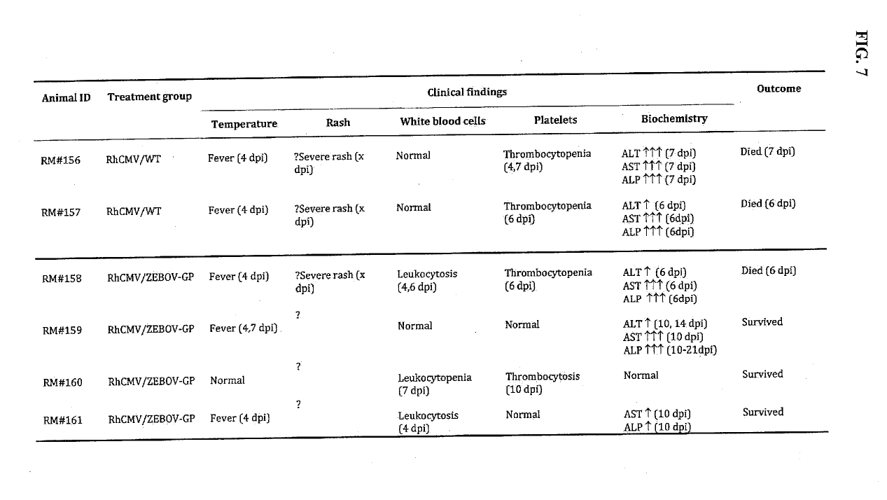

To determine whether immunity induced by RhCMV/ZEBOV-GP was able to protect animals from lethal ZEBOV disease, the RMs were then challenged with a lethal 1,000 ffu dose of low passage (7U) ZEBOV virus at day 0. RMs were monitored twice daily, and physical exams and blood draws were conducted on day 0, 4, 7, 14, 21, 28, and 35 post-ZEBOV challenge (FIG. 3A). Clinical findings are presented in FIGS. 4A-I and FIG. 7. Three of 4 RhCMV/ZEBOV-GP vaccinated RMs survived ZEBOV challenge (RM#159, RM#160 and RM#161) indicating that vaccination had induced a protective immune response against ZEBOV. Two of the three protected animals were febrile (>1° C. above baseline) at day 4 post-challenge, but all animals returned to normal by day 10. Transient low level viremia was observed in one animal (RM#159) at a single timepoint (day 7 post-challenge) (5, 623 TCID₅₀/ml compared to 1.78×10⁷ and 1.78×10⁸ TCID₅₀/ml in controls), but viremia was undetectable in the remaining animals (RM#160 and RM#161). A transient increase in levels of AST, ALT and ALP was also observed in RM#159 consistent with the day 7 viremia being associated with self-limiting mild liver disease. Although within normal range (ie., <30% decrease from baseline), RM#159 also showed a transient drop in platelet levels at day 7. Given the absence of viremia in RM#160 and RM#161, the clinical scores observed in these animals may result from host inflammatory response mechanisms associated with ZEBOV control. The 2 RM controls (RM#156 and RM#157), which received RhCMV-WT were both febrile at day 4 post-challenge, and then rapidly developed ZEBOV-associated disease, reaching a predetermined clinical humane endpoint by days 6 and 7 post challenge. At this point, clinical disease progression was considered irreversible and animals were humanely euthanized in accordance with IACUC protocols. A single animal from the vaccinated group (RM#158) showed a similar disease progression as controls, and was euthanized on day 6 post-challenge. Despite similar disease progression, the kinetics of viremia in RM#158 were delayed, being 1- and 2-logs lower than control animals at 4 days post-challenge, suggesting that RhCMV/ZEBOV-GP vaccination may have been still providing some low, partial level of protection in this animal. By day 6 post-challenge ZEBOV levels in blood and tissues were comparable in RM#158 and controls. At this time, severe thrombocytopenia was present all 3 unprotected animals. Animals also showed highly elevated levels of AST, ALT and ALP indicative of ZEBOV-associated liver damage. Presence of macular cutaneous rash/petechia over multiple areas in all animals was consistent with hemorrhagic manifestations of ZEBOV disease.

Given that the normal immunological phenotype for a RhCMV-based vector is heavily biased towards cellular ‘effector’ T cell memory with minimal antibody production^{20,32,33,36}, the induction of high levels of anti-GP antibodies by the RhCMV/ZEBOV-GP, with an absence of detectable GP-specific T cells, was a surprising observation. Although the present study was not powered for identification of immune correlates of protection, the magnitude of total anti-GP IgG level corresponded to differences in protection between the RhCMV/ZEBOV-GP vaccinated animals [need to confirm day 0 level of RM#158], with the single non-protected vaccinated animal (RM#159) having consistently lower levels of total IgG against ZEBOV throughout the vaccine phase. Protection afforded by the recombinant vesicular stomatitis virus (VSV)-based ZEBOV vaccine currently undergoing Phase I human trials³⁷ similarly corresponds to antibody level, with high levels of total anti-GP antibodies being a consistent predictor of survival³⁸⁻⁴⁰. A recent cellular depletion study in NHPs has confirmed that antibody rather than cellular GP-specific immune responses are of primary importance for rVSV-mediated protection⁴¹, The rapid ‘consumption’ of anti-GP antibodies that we observed in vaccinated animals at day 4 post-challenge (FIG. 3C) has been seen before in ZEBOV immune animals following ZEBOV challenge³⁸. Given the speed of decline (within 4 days of challenge) and the absence of leukocytopenia, this drop in antibody levels is consistent with antibody consumption as a consequence of its involvement in ZEBOV control. In the absence of detectable GP-specific cellular immune responses in any of the RhCMV/ZEBOV-GP vaccinated animals prior to ZEBOV challenge (FIG. 2A), the decrease of GP-antibodies to below the threshold of detection level in only the single vaccinated animal that died from ZEBOV disease is persuasive of protection being afforded by a predominantly antibody-mediated mechanism. Additional studies powered to identify correlates of protection, as well as the use of depletion to directly ascertain protective mechanism will be necessary to confirm whether protection from RhCMV/ZEBOV-GP is primarily antibody-mediated.

The mechanism responsible for this unique antibody-biased immune response associated with RhCMV/ZEBOV-GP vaccination is an area of ongoing study. We hypothesize that the phenotype results from the expression of GP under control of the Rh112 L promoter, a promoter that is expressed a L times of RhCMV infection. Expression at this time coincides with the expression of multiple CMV-encoded immunoevasins that downregulate MHC expression. In our model, the inability to present the heterologous target antigen to T cells via the canonical MHC pathway would shift immune responses away from cellular towards humoral immunity. This also raises the possibility for rational design of vaccines that can direct the target pathogen-specific immune response towards either antibody production with diminished T cell responses (by use of a promoter expressed at L times), or towards T cell responses with diminished antibody responses (by use of a promoter expressed at IE and E times). By use of promoters that are intermediate in their kinetics of expression (E-L), a balanced antibody and T cell response could be achieved. Alternatively a mixture of vaccines using different kinetic classes of promoters to drive target antigen expression, either within distinct vaccines or within the same vaccine construct, would enable a balanced antibody and T cell response to be achieved.

In summary, the present study shows that the protective immunity of CMV vectors initially suggested in mouse studies using a MCMV-based vaccine translates into protective efficacy using RhCMV-based vectors in the ‘gold standard’ NHP ZEBOV challenge model. A primary goal of our studies is development of a ‘self-disseminating’ vaccine to target inaccessible African apes in the wild, both to prevent transmission of ebolavirus into the human population, but also to prevent ZEBOV-mediated eradication of these ape populations. Future studies will need to explore further the impact of CMV promoter usage on immune response characteristics, with the aim of inducing both cellular as well as substantial levels of humoral ZEBOV-specific immunity. In our study, RhCMV/ZEBOV-GP vaccine ‘take’ was clearly unperturbed by pre-existing immunity against the vector, as all RMs used in the present study were CMV seropositive at the time of vaccination. Immunity following animal-to-animal spread of the vector will now need to be investigated. Results from the present study also support the potential for development of CMV as prophylactic vaccine for ebolavirus in humans potentially using an attenuated or replication-deficient CMV platform. Ad-based and VSV-based ebolavirus vaccines differ in their modes of protection—the former being primarily associated with cellular immunity, whilst VSV is antibody-mediated. The current study suggests the unique possibility of being able to recruit both arms of the immune response together against ebolavirus infection in a single CMV vaccine by combined use of differential promoter usage.

### FIG. 1. Construction and Characterization of RhCMV Vectors Engineered to Express EBOV (Zaire) GP (Designated RhCMV/ZEBOV-GP).

FIG. 1A. Schematic representation of RhCMV/ZEBOV-GP showing predicted topology. A codon-optimized full-length ZEBOV glycoprotein (GP) (Zaire) was inserted within the RhCMV genome (68.1) to replace the endogenous Rh112 (pp65b). This approach places GP under the control of the endogenous RhCMV Rh112 promoter. FIG. 1B. Multi-step growth analysis of RhCMV/ZEBOV-GP. RFs were infected at a MOI of 0.01 with either RhCMV/WT, RhCMV/ZEBOV-GP[2-8] or RhCMV/ZEBOV-GP[6-1]. Supernatant was collected at days indicated post infection and titered in a TCID₅₀ assay. The assay was performed in triplicate and standard deviation is shown. FIG. 1C. Western analysis of RhCMV/ZEBOV-GP in RFs showing stable expression of ZEBOV GP until at least passage 7. Western blot analysis of RhCMV/ZEBOV-GP infected cell lysates based on V5 epitope tag and anti-GP MAb detection, showed that GP was expressed at high levels, and that expression was stable over multiple passages (passage 7). V5 activity was observed against 3 bands as predicted [110 kDa preGPer (not shown); 160 kDa preGP (not shown) and 26 kDa GP2]. Anti-GP MAb was used to detect GP1 (140 kDa) as the V5 tag is localized at the carboxyl-terminus of preGP.

### FIG. 2. RhCMV/ZEBOV-GP Expresses ZEBOV-GP at Late Times of Replication.

FIG. 2. Western analysis RhCMV/ZEBOV-GP in RFs showing ZEBOV GP expression at L times of replication. RFs were infected at a MOI of 0.01 with either RhCMV/WT, RhCMV/ZEBOV-GP[2-8] or RhCMV/ZEBOV-GP[6-1]. Cell lysates were collected at days indicated post infection and analysed by western blot. Accumulation of ZEBOV-GP was compared to accumulation of viral proteins known to be expressed with IE (IE-1) and L (pRh112) kinetics.

FIGS. 3A-3C. RhCMV/ZEBOV-GP Induces High Levels of Antibodies Against ZEBOV-GP, with Absence of ZEBOV-GP Directed T Cell Responses in RMs.

FIG. 3A. Schematic showing timeline of ‘vaccine’ and ‘challenge’ phases and sampling schedule. FIG. 3B. Time course of CD4⁺ and CD8⁺ T cell responses against IE-1, pRh112 (pp65b) and ZEBOV GP. T cells were analysed by ICS following incubation with overlapping peptide pools in the presence of BFA. Levels of responding cells (TNFα and IFNγ double-positive) in individual RMs are shown at times indicated. T cell responses against endogenous RhCMV antigens (IE-1 and Rh112) were observed in all animals, while no responses against ZEBOV GP were detected at any time. FIG. 3C. Time course of antibody responses against ZEBOV GP. Total IgG antibody levels against ZEBOV GP were measured by ELISA at times indicated. Antibodies were detected after the initial RhCMV/ZEBOV-GP vaccination, and then increased further following the boost at day −28. The drop in antibody levels observed at 4 days post-challenge is consistent with antibody consumption during control of ZEBOV infection.

### FIGS. 4A-4I. Clinical Parameters in RhCMV/WT and RhCMV/ZEBOV-GP Vaccinated Animals.

Changes in various clinical parameters were measured over the duration of the study. (A) Kaplan-Meier survival curves, (B) temperature, (C) daily clinical scores, (D) viremia, (E) WBCs, (F) platelets, (G) AST levels, (H) ALT levels and (I) ALP levels.

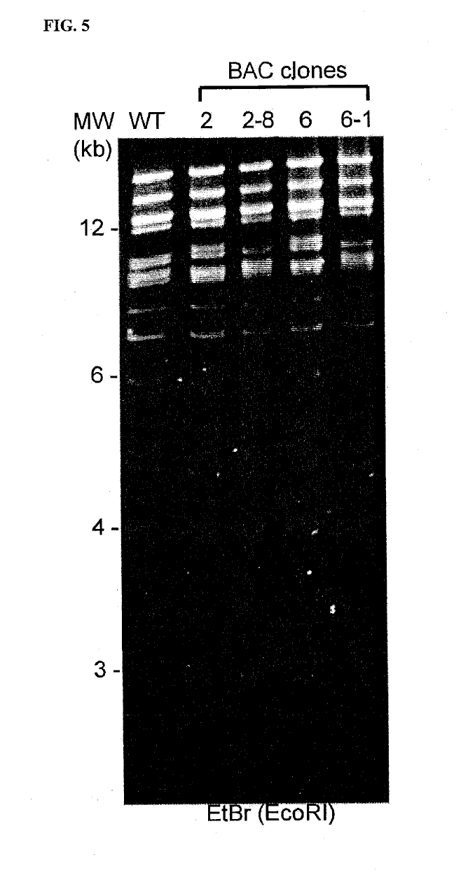

### FIG. 5.

Genomic characterization of RhCMV/ZEBOV-GP BAC. DNA from two independent clones of RhCMV/ZEBOV-GP [2-8 and 6-1] were digested with EcoRI followed by electrophoresis. The comparable digest pattern between RhCMV/ZEBOV-GP [6-1] and RhCMV/WT BAC shows the lack of any gross genomic rearrangement. A band shift was observed in the RhCMV/ZEBOV-GP BAC digest and was localized to X region.

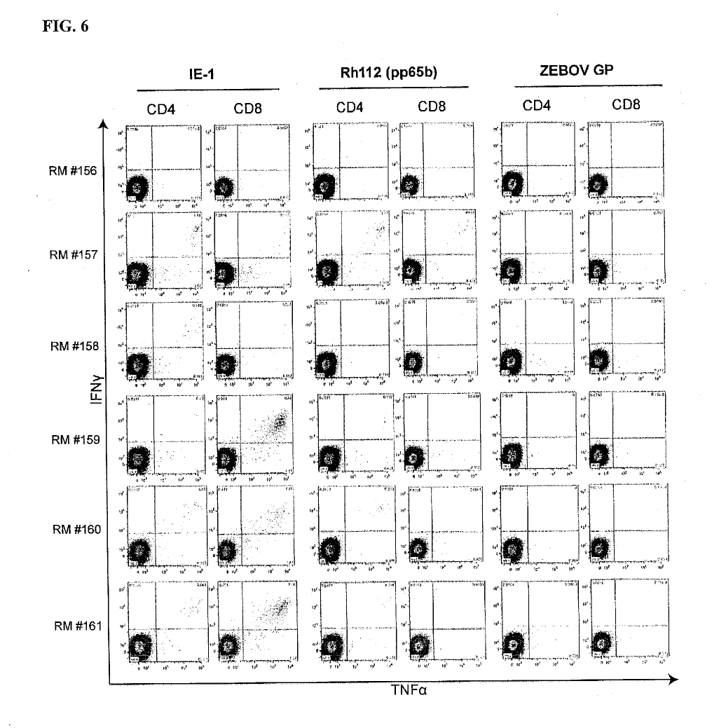

### FIG. 6.

Flow cytometry showing original dot-plot data presented graphically in FIG. 3.

### FIG. 7. Clinical Findings During ZEBOV Challenge Phase.

Fever was defined as ≧1° C. above baseline. Mild rash was defined as areas of petechiae covering less that 10% of the skin, moderate rash was defined as areas of petechiae covering 10-40% of the skin and severe rash was defined as areas of petechiae covering >40% of the skin. Leukocytopenia and thrombocytopenia were defined as a >30% decrease in numbers of WBCs and platelets, respectively. Leukocytosis and thrombocytosis were defined as a twofold or greater increase in numbers of WBCs and platelets above baseline levels, where WBC count >11,000. Elevated ALT, AST and ALP levels were defined as: ↑ (>2-fold; <4-fold increase), ↑↑ (>4-fold; <5-fold increase) and ↑↑↑ (>5-fold increase).

### Supplemental Table 1.

Combined Sanger and NGS Sequencing of BACs and reconstituted RhCMV/ZEBOV-GP (Clone 2-8 and 6-1).

### Supplemental Table 2.

Total and neutralizing antibody levels pre- and post-ZEBOV challenge.

### Methods

Methods and any associated references are available in the online version of the paper at http://www.nature.com/nm/.

### Animal Ethics Statement.

The study was approved by the Institutional Animal Care and Use Committee (IACUC) at the Rocky Mountain Laboratories (RML) (ASP#2014-020E). RML is accredited by the American Association for Accreditation of Laboratory Animal Care (Public Health Service/Office of Laboratory Animal Welfare Assurance #A4149-01). All animal work was performed in strict accordance with recommendations detailed in the ‘Guide for the Care and Use of Laboratory Animals’.⁴² Procedures were performed after ketamine-induced anesthesia by trained personnel under direct supervision of veterinarians. All efforts were made to minimize animal suffering based on recommendations of the working group report chaired by Sir David Weatherall “The use of non-human primates in research”.⁴³ Rhesus macaques (Macaca mulatta) were housed under high containment at RML in individual adjoining primate cages enabling social interactions between animals. Humidity, temperature and light (12 hour light/dark cycles) were carefully regulated. Environmental enrichment was provided for animals by providing access to commercial toys. Animals were fed with commercial monkey chow, treats and fruit twice daily. Water was available ad libitum. Animals were monitored twice daily for the duration of the study (during both vaccine and ZEBOV challenge phases). Early endpoint criteria using score parameters approved by the RML IACUC were used to avoid unnecessary animal suffering (see below). Once the endstage of disease had been reached based on early endpoint criteria, animals were humanely euthanized.

### Challenge Virus.

We used a low passage ZEBOV (strain Mayinga) as the challenge virus for these studies⁴⁴. ZEBOV was prepared in African green monkey kidney (Vero E6) cells. For stock production, we removed cellular debris by centrifugation, followed by aliquoting and storage of stocks under liquid nitrogen until use. EBOV utilizes transcriptional editing to regulate levels of soluble GP (sGP) compared to the transmembrane virion-associated peplomer form (GP_{1,2}) by insertion of a non-templated adenine residue within a 7 uridine (poly-U) RNA-editing tract of the GP gene.⁴⁵ Recombinant EBOVs containing a genomically-encoded 8U tract are also known to accumulate during EBOV passage in Vero E6 cells, but are rapidly selected against following in vivo passage.^{46,47} The ZEBOV challenge virus stock used in the present study was derived by limited in vitro passage in Vero E6 cells, and was confirmed to be primarily of the 7U form by reverse transcription (RT)-PCR followed by DNA Sanger sequencing. All infectious work using ZEBOV was performed under biosafety level 4 (BSL-4) containment at the Integrated Research Facility in the RML, Division of Intramural Research, National Institute of Allergy and Infectious Diseases (NIAID), National Institutes of Health (NIH), Hamilton, Mont., USA.

### Vaccine Vectors.

We constructed RhCMV/ZEBOV-GP essentially as previously described⁴⁸ by using E/T linear recombination to manipulate the parental RhCMV strain 68-1 genome cloned within a bacterial artificial chromosome (BAC) (designated pRhCMVIBAC-Cre).⁴⁹ A previously constructed codon-optimized version of GP (optZGP) from ZEBOV (Mayinga strain 76; Accession number AF086833) was used as the target antigen³⁴. The optZGP was codon-optimized by using the most frequent codons found in mammalian proteins, such that 70% (compared to 36% of non-optimized GP) of the codons present in optZGP are either the first or second most abundantly used mammalian codons.³⁴ As a strategy to increase stability of optZGP expression, the optZGP open reading frame (ORF) was inserted within the RhCMV genome to replace the non-essential endogenous RhCMV Rh112 (pp65b) ORF (nucleotide positions 111,240 to 112,868 of RhCMV; see schematic FIG. X).⁵⁰ This strategy places optZGP under control of the Rh112 promoter and also uses the endogenous Rh112 poly-adenylation signal sequence. Rh112 has been shown to be non-essential for infection or persistence of RhCMV in healthy seropositive or seronegative rhesus macaques.³⁵ We also epitope tagged optZGP at the carboxy terminus with a V5 epitope tag to facilitate analysis of protein expression.⁵¹ The optZGP was cloned into a recombination cassette as a necessary requirement prior to E/T linear recombination. Within the recombination cassette, a FRT-flanked kanamycin resistance (Kan^{R}) marker was present immediately down-stream from optZGP ORF, which enabled selection of recombinant BAC clones on the basis of kanamycin resistance. We subsequently removed the Kan^{R} marker by arabinose induction of flp-recombinase, and recombinants were screened on the basis of kanamycin sensitivity. We analyzed recombinant RhCMV/ZEBOV-GP BAC clones by restriction enzyme digestion (FIG. 5), as well as direct Sanger-based sequence analysis of the optZGP ORF (Supplemental Table 1). Recombinant RhCMV/ZEBOV-GP viruses were reconstituted by transfection of BAC DNA into RhCMV permissive rhesus fibroblasts (RFs) followed by serial passage to enable Cre-recombinase mediated excision of the BAC cassette.⁴⁹ We confirmed stable optZGP expression in RhCMV/ZEBOV-GP vectors over at least 7 passages by western analysis of infected cell lysates using a monoclonal antibody directed against ZEBOV GP₁ and against the V5 epitope tag (Invitrogen; used at 1:2000) (FIG. 1). Multi-step growth analysis of the RhCMV/ZEBOV-GP was performed as previously described (FIG. 6).⁴⁸ Next generation sequencing (NGS) of BACs and reconstituted viruses was used for complete genome sequence characterization of the RhCMV/ZEBOV-GP vectors (Supplemental Table 1).

### Rhesus Macaques.

We used 6 captivity-bred adult male and female rhesus macaques of Indian genetic background. Animals were confirmed to be RhCMV seropositive by ELISA before initiation of the study (resulting from natural RhCMV infection). Animals were filovirus-naïve and were also free of cercopithicine herpesvirus 1, simian T-lymphotropic virus type 1, D-type simian retrovirus and simian immunodeficiency virus. We assigned animals to either RhCMV/ZEBOV-GP vaccine (n=4) or parental wild-type (WT) RhCMV control (n=2) groups with an aim of achieving a relatively equal distribution based on sex and age. On day −112, the vaccine group received a single sub-cutaneous (s.c.) bolus of a mixture of two independent clones of the RhCMV/ZEBOV-GP construct [5×10⁶ plaque forming units (pfu)/construct]. The RhCMV WT control group received a single 1×10⁷pfu s.c. inoculation of parental RhCMV WT (clone 68-1).⁴⁹ Animals were boosted with either RhCMV/ZEBOV-GP or RhCMV WT at 20 week 12 (day −28). We collected blood samples at times indicated over the pre-challenge 112 day period (vaccine phase) (FIG. 2). Peripheral blood mononuclear cells (PBMCs) and plasma were prepared from blood by centrifugation on a histopaque gradient (Sigma) and assayed as detailed below. On day 0 (112 days post-vaccination), we challenged all animals with a lethal dose of 1,000 focus forming units (ffu) of ZEBOV by intra-muscular (i.m.) administration at two anatomical locations (left and right caudal thigh). We continued to monitor animals twice daily for clinical signs of disease. Disease progression was assessed based on pre-established endpoints (described below), and animals were humanely euthanized when clinical signs indicated onset of terminal disease. Blood samples were collected at times indicated (FIG. 2) over the 35 day post-challenge period.

### Clinical Score.

We monitored animals twice daily over the entire study period (vaccine and ZEBOV challenge phases) using clinical score criteria approved by the RML IACUC. Assessment was based on the following criteria: i) general appearance, ii) condition of skin and fur, nose, mouth, eyes and head, iii) level of food intake, iv) quality and output of feces and urine, v) respiration, and vi) locomotor activity. Scores were recorded in a daily observation log, and animals were humanely euthanized when the total score reached 35. Euthanasia was also performed if any of the following signs were observed: i) impaired movement preventing access to food or water, ii) excessive weight loss, iii) loss of mental alertness, iv) difficulty in breathing, or v) prolonged inability to maintain upright posture.

### Hematology and Serum Chemistry.

We used a HemaVet® 950FS laser-based hematology analyzer (Drew Scientific) to analyze the following blood parameters in 20 μl volumes of EDTA-treated blood: i) total white blood cell count, ii) lymphocyte, platelet, reticulocyte and red blood cell counts, iii) hemoglobin, iv) hematocrit values, and v) mean corpuscular volume and hemoglobin concentrations. Serum chemistry was analyzed using a Piccolo Xpress Chemistry Analyzer using Piccolo General Chemistry 13 Panel discs (Abaxis).

### Plasma Cytokine Levels.

We diluted rhesus macaque plasma samples 1:2 in serum matrix prior to analysis of plasma cytokine levels (IL-1Ra, IL-4, IL-6, IL-8, IL-12/23p40, IL-5, IL-17, soluble CD40L, IFNγ, MCP-1 and TNFα) by using Milliplex Non-Human Primate Magnetic Bead panels according to the manufacturer's (Millipore) protocol. Post-challenge plasma samples were inactivated by γ-irradiation (5 Mrad) prior to removal from BSL-4 containment under standard RML operating procedures as approved by the RML IBC.

### Viral Loads.

We quantitated ZEBOV blood levels by using quantitative RT-PCR (qRT-PCR) as previously described.⁵² We also measured levels of infectious ZEBOV by using standard virus titration⁴¹, followed by calculation of 50% tissue culture infectious dose (TCID₅₀) using the method of Reed and Muench.⁵³ Tissues were homogenized prior to analysis.

### Intracellular Cytokine Staining Analysis of T Cells.

Frequencies of CD4⁺ and CD8⁺ T cells directed against the ZEBOV (Mayinga) GP target antigen, as well as RhCMV immediate early 1 (IE1) protein were determined during the vaccine phase by intracellular cytokine staining (ICS) as previously described.^{41,54} For stimulation, PBMC (1-2×10⁶ cells/well) were incubated in vitro with peptide pools (1 μg/ml final concentration) of overlapping peptides (11-mer with 5 amino acid overlap) representing each of the target ORFs. Incubation without antigen served as a background control. After 1 hour, brefeldin A (10 μg/ml) was added and cells were incubated for an additional 14 hours. Cells were surface stained using the following mAbs in indicated combinations: CD3, CD4 (eBioscience) and CD8β (Beckman Coulter). Cells were fixed and permeabilized according to manufacturer's recommendations (BioLegend) prior to staining for intracellular staining using mAbs against Ki67 (BD) and IFNγ and TNFα. Polychromatic flow cytometric analysis was performed on a LSR II (BD Biosciences), and data was analyzed by using FlowJo software (version 10; Tree Star, Inc.). Response frequencies were determined by subtracting background and then averaging background subtracted responses.

### Enzyme-Linked Immunosorbent Assay (ELISA).

We measured total IgG antibody responses to RhCMV/ZEBOV-GP by ELISA using ZEBOV-GPΔ™ as a source of antigen, as previously described⁵⁵. Analysis was performed as BSL-2. We show the end-point dilution titer (using a 4-fold dilution series). Analysis was performed at BSL-2. Post-challenge plasma samples were inactivated by γ-irradiation (5 Mrad) before removal from BSL-4 containment under standard RML operating procedures as approved by the RML Institutional Biosafety Committee (IBC). Samples were deemed positive when the OD value was higher than the mean plus 3 standard deviations of negative (RhCMV WT) sera.⁵⁶

### Neutralization Assay.

We analyzed plasma collected from animals at times indicated for ability to neutralize ZEBOV in an in vitro neutralization assay.³⁴ Briefly, heat inactivated sera was serially diluted in DMEM, and then mixed 1:1 with ZEBOV expressing the EGFP reporter (200 FFU/well). After incubation at 37° C. for 60 minutes, we transferred 20 μl of the mixture onto subconfluent Vero E6 cells in a 96-well plate format and incubated for 30 minute at 37° C. Following addition of 1801 of DMEM supplemented with 1.5% carboxymethyl cellulose and 5% FBS, we cultured the cells for 4 days at 37° C. The cells were then washed with PBS and fixed in 10% neutral buffered formalin overnight under BSL-4 conditions. We then processed plates under BSL-4 conditions by conventional methods. We show the sera dilutions that resulted in 50% reduction in EGFP-positive cells following infection of Vero E6 cells with EGFP-labeled viruses.

### Statistical Analysis.

Tests used for analysis are indicated in individual figure legends. One-way ANOVA with Bonferroni correction was used to compare immune responses and disease parameters between RhCMV/ZEBOV-GP and control groups. Statistical significance was determined at a level of 0.05. Kaplan-Meier log-rank test was used to compare survival rates between groups in ZEBOV challenge studies. All analysis was performed using Prism GraphPad Software (Version 5.0d).

### Acknowledgements

We thank Dr P. Barry (University of California at Davis, Calif., USA) for providing the pRhCMV (68.1) RhCMV BAC, and Dr D. Court (NCI-Frederick, Md.) for providing the E/T-based recombination system, and, Dr T. Shenk and Dr W. Britt for providing antibodies.

Although illustrative embodiments of the invention have been disclosed in detail herein, with reference to the accompanying drawings, it is understood that the invention is not limited to the precise embodiments shown and that various changes and modifications can be effected therein by one skilled in the art without departing from the scope of the invention as defined by the appended claims and their equivalents.

### REFERENCES

1. Ebola virus disease in West Africa—the first 9 months of the epidemic and forward projections. N Engl J Med 371, 1481-1495 (2014).
2. Rizkalla, C., Blanco-Silva, F. & Gruver, S. Modeling the impact of Ebola and bushmeat hunting on Western Lowland Gorillas. EcoHealth 4, 151-155 (2007).
3. Groseth, A., Feldmann, H. & Strong, J. E. The ecology of Ebola virus. Trends in microbiology 15, 408-416 (2007).
4. Leroy, E. M., et al. Multiple Ebola virus transmission events and rapid decline of central African wildlife. Science 303, 387-390 (2004).
5. Rouquet, P., et al. Wild animal mortality monitoring and human Ebola outbreaks, Gabon and Republic of Congo, 2001-2003. Emerg Infect Dis 11, 283-290 (2005).
6. Leroy, E. M., et al, Human Ebola outbreak resulting from direct exposure to fruit bats in Luebo, Democratic Republic of Congo, 2007. Vector Borne Zoonotic Dis 9, 723-728 (2009).
7. Tsuda, Y., et al. A replicating cytomegalovirus-based vaccine encoding a single Ebola virus nucleoprotein CTL epitope confers protection against Ebola virus. PLoS Neglected Tropical Diseases 5, e1275 (2011).
8. Legrand, J., Grais, R. F., Boelle, P. Y., Valleron, A. J. & Flahault, A. Understanding the dynamics of Ebola epidemics. Epidemiol Infect 135, 610-621 (2007).
9. Frontieres, M. S. Filovirus Haemorrhagic Fever Guideline. (2008).
10. Warfield, K. L, et al. Vaccinating captive chimpanzees to save wild chimpanzees. Proceedings of the National Academy of Sciences of the United States of America 111, 8873-8876 (2014).
11. Pourrut, X., et al. Large serological survey showing cocirculation of Ebola and Marburg viruses in Gabonese bat populations, and a high seroprevalence of both viruses in Rousettus aegyptiacus. BMC Infect Dis 9, 159 (2009).
12. Formenty, P., et al. Ebola virus outbreak among wild chimpanzees living in a rain forest of Cote d'lvoire. J Infect Dis 179 Suppl 1, S120-126 (1999).
13. Georges, A. J., et al. Ebola hemorrhagic fever outbreaks in Gabon, 1994-1997: epidemiologic and health control issues. The Journal of infectious diseases 179 Suppl 1, S65-75 (1999).
14. Walsh, P. D., et al. Catastrophic ape decline in western equatorial Africa. Nature 422, 611-614 (2003).
15. Bermejo, M., et al. Ebola outbreak killed 5000 gorillas. Science 314, 1564 (2006).
16. Ryan, S. J. & Walsh, P. D. Consequences of non-intervention for infectious disease in African great apes. PLoS One 6, e29030 (2011).
17. Walsh, P. D., et al. The IUCN Red List of Threshold Species. Version 2014.2. Vol. 2014 (2008).
18. Murthy, S., et al. Absence of frequent herpesvirus transmission in a nonhuman primate predator-prey system in the wild. J Virol 87, 10651-10659 (2013).
19. Kern, E. R. Pivotal role of animal models in the development of new therapies for cytomegalovirus infections. Antiviral Res 71, 164-171 (2006).
20. Hansen, S. G., et al. Profound early control of highly pathogenic SIV by an effector memory T-cell vaccine. Nature 473, 523-527 (2011).
21. Jarvis, M. A., Hansen, S. G., Nelson, J. A., Picker, L J. & Frueh, K. Vaccine vectors using the unique biology and immunology of cytomegalovirus. in Cytomegaloviruses: From Molecular Pathogenesis to Intervention, Vol. II (ed. Reddehase, M. J.) (Caister Academic Press, 2012).
22. Redwood, A. J., et al. Use of a murine cytomegalovirus K181-derived bacterial artificial chromosome as a vaccine vector for immunocontraception. Journal of virology 79, 2998-3008 (2005).
23. Tierney, R., et al. A single-dose cytomegalovirus-based vaccine encoding tetanus toxin fragment C induces sustained levels of protective tetanus toxin antibodies in mice. Vaccine 30, 3047-3052 (2012).
24. Klyushnenkova, E. N., et al. A cytomegalovirus-based vaccine expressing a single tumor-specific CD8+ T-cell epitope delays tumor growth in a murine model of prostate cancer. J Immunother 35, 390-399 (2012).
25. Ross, S. A., et al. Cytomegalovirus reinfections in healthy seroimmune women. J Infect Dis 201, 386-389 (2010).
26. Farroway, L. N., et al. Transmission of two Australian strains of murine cytomegalovirus (MCMV) in enclosure populations of house mice (Mus domesticus). Epidemiol Infect 133, 701-710 (2005).
27. Boppana, S. B., Rivera, L. B., Fowler, K. B., Mach, M. & Britt, W. J. Intrauterine transmission of cytomegalovirus to infants of women with preconceptional immunity. N Engl J Med 344, 1366-1371 (2001).
28. Hansen, S. G., et al. Evasion of CD8+ T cells is critical for superinfection by cytomegalovirus. Science 328, 102-106 (2010).
29. Reed, D. S. & Mohamadzadeh, M. Status and challenges of filovirus vaccines. Vaccine 25, 1923-1934 (2007).
30. Hoenen, T., Groseth, A. & Feldmann, H. Current ebola vaccines. Expert Opin Biol Ther 12, 859-872 (2012).
31. Stanley, D. A., et al. Chimpanzee adenovirus vaccine generates acute and durable protective immunity against ebolavirus challenge. Nature medicine 20, 1126-1129 (2014).
32. Hansen, S. G., et al. Effector memory T cell responses are associated with protection of rhesus monkeys from mucosal simian immunodeficiency virus challenge. Nature medicine 15, 293-299 (2009).
33. Hansen, S. G., et al. Immune clearance of highly pathogenic SIV infection. Nature 502, 100-104 (2013).
34. Richardson, J. S., et al. Enhanced protection against Ebola virus mediated by an improved adenovirus-based vaccine. PLoS One 4, e5308 (2009).
35. Malouli, D., et al. Cytomegalovirus pp65 limits dissemination but is dispensable for persistence. J Clin Invest (2014).
36. Bilello, J. P., et al. Vaccine protection against simian immunodeficiency virus in monkeys using recombinant gamma-2 herpesvirus. Journal of virology 85, 12708-12720 (2011).
37. Cohen, J. Infectious Disease. Ebola vaccines racing forward at record pace. Science 345, 1228-1229 (2014).
38. Qiu, X., et al. Sustained protection against Ebola virus infection following treatment of infected nonhuman primates with ZMAb. Sci Rep 3, 3365 (2013).
39. Wong, G., et al. Immune parameters correlate with protection against ebola virus infection in rodents and nonhuman primates. Science translational medicine 4, 158ra146 (2012).
40. Jones, S. M., et al. Live attenuated recombinant vaccine protects nonhuman primates against Ebola and Marburg viruses. Not Med 11, 786-790 (2005).
41. Marzi, A., et al. Antibodies are necessary for rVSV/ZEBOV-GP-mediated protection against lethal Ebola virus challenge in nonhuman primates. Proceedings of the Notional Academy of Sciences of the United States of America 110, 1893-1898 (2013).
42. Animals, C.f.t.U.o.t.G.f.t.C.a.U.o.L. Guide for the Care and Use of Laboratory Animals. (Institute for Laboratory Animal Research, 2011).
43. Weatherall, W.G.R.c.b.S.D. The Use of non-human primates in research. (2006).
44. Blaney, J. E., et al. Antibody quality and protection from lethal Ebola virus challenge in nonhuman primates immunized with rabies virus based bivalent vaccine. PLoS pathogens 9, e1003389 (2013).
45. Sanchez, A., Trappier, S. G., Mahy, B. W., Peters, C. J. & Nichol, S. T. The virion glycoproteins of Ebola viruses are encoded in two reading frames and are expressed through transcriptional editing. Proceedings of the National Academy of Sciences of the United States of America 93, 3602-3607 (1996).
46. Volchkova, V. A., Dolnik, O., Martinez, M. J., Reynard, O. & Volchkov, V. E. Genomic RNA editing and its impact on Ebola virus adaptation during serial passages in cell culture and infection of guinea pigs. The Journal of infectious diseases 204 Suppl 3, S941-946 (2011).
47. Kugelman, J. R., et al. Ebola virus genome plasticity as a marker of its passaging history: a comparison of in vitro passaging to non-human primate infection. PloS one 7, e50316 (2012).
48. Rue, C. A., et al. A cyclooxygenase-2 homologue encoded by rhesus cytomegalovirus is a determinant for endothelial cell tropism. Journal of virology 78, 12529-12536 (2004).
49. Chang, W. L. & Barry, P. A. Cloning of the full-length rhesus cytomegalovirus genome as an infectious and self-excisable bacterial artificial chromosome for analysis of viral pathogenesis. J Virol 77, 5073-5083 (2003).
50. Hansen, S. G., Strelow, L I., Franchi, D. C., Anders, D. G. & Wong, S. W. Complete sequence and genomic analysis of rhesus cytomegalovirus. Journal of Virology 77, 6620-6636 (2003).
51. Minde, D. P., Halff, E. F. & Tans, S. J. Designing disorder. Tales of the unexpected tails. Intrinisically Disordered Proteins 1, e26790-26791 (2013).
52. Marzi, A., et al. Vesicular stomatitis virus-based ebola vaccines with improved cross-protective efficacy. The Journal of infectious diseases 204 Suppl 3, S1066-1074 (2011).
53. Reed, L. J. & Muench, H. A simple method of estimating fifty percent endpoints. American Journal of Hygeine 27, 493-497 (1938).
54. Haberthur, K., et al. Genome-wide analysis of T cell responses during acute and latent simian varicella virus infections in rhesus macaques. Journal of virology 87, 11751-11761 (2013).
55. Nakayama, E., et al. Enzyme-linked immunosorbent assay for detection of filovirus species-specific antibodies. Clinical and vaccine immunology: CVI 17, 1723-1728 (2010).
56. Qiu, X., et al. Mucosal immunization of cynomolgus macaques with the VSVDeltaG/ZEBOVGP vaccine stimulates strong ebola GP-specific immune responses. PLoS One 4, e5547 (2009).

1-44. (canceled)

45. Use of a recombinant herpesvirus-based vector to provide an immune response in a subject which is biased towards an antibody response, the vector comprising a nucleic acid sequence encoding a heterologous antigen and a promoter for controlling the expression of the antigen, in which the promoter is expressed at L times.

46. Use as claimed in claim 45, in which the herpesvirus-based vector is a CMV-based vector.

47. Use as claimed in claim 46, in which the CMV-based vector is selected from the group consisting of: Human CMV (HCMV), Simian CMV (SCCMV), Rhesus CMV (RhCMV), Chimpanzee CMV (CCMV) Murine CMV (MCMV) and Gorilla CMV (GCMV).

48. Use as claimed in claim 45, wherein the heterologous antigen is a pathogen-specific antigen.

49. Use as claimed in claim 45, wherein the heterologous antigen is a human pathogen-specific antigen.

50. Use as claimed in claim 49, wherein the human pathogen-specific antigen is a viral antigen.

51. Use as claimed in claim 50, in which the viral antigen is selected from the group consisting of: human immuno-deficiency virus, simian immuno-deficiency virus, Kaposi's sarcoma-associated herpesvirus, Herpes simplex virus 1, Herpes simplex virus 2, Epstein Barr virus, hepatitis B virus, human papillomavirus, influenza virus, monkeypox virus, West Nile virus, Chikungunya virus, Ebola virus, hepatitis C virus, poliovirus, dengue virus, herpes virus B, Marburg virus, SARS virus, and MERS virus.

52. Use as claimed in claim 50, wherein the viral antigen is a viral protein, an epitope or antigenic fragment thereof.

53. Use as claimed in claim 45, wherein the heterologous antigen is a bacterial antigen.

54. Use as claimed in claim 53, wherein the bacterial antigen is a bacterial protein, an epitope or antigenic fragment thereof.

55. A method of preventing or treating an infectious disease by providing an immune response in a subject which is biased towards an antibody cell response, the method comprising administration of a recombinant herpesvirus-based vector, the vector comprising a nucleic acid sequence encoding a heterologous antigen and a promoter for controlling the expression of the antigen, in which the promoter is expressed at L times.

56. A method as claimed in claim 55, in which the herpesvirus-based vector is a CMV-based vector.

57. A method as claimed in claim 56, in which the CMV-based vector is selected from the group consisting of: Human CMV (HCMV), Simian CMV (SCCMV), Rhesus CMV (RhCMV), Chimpanzee CMV (CCMV) Murine CMV (MCMV) and Gorilla CMV (GCMV).

58. A method as claimed in claim 55, wherein the heterologous antigen is a pathogen-specific antigen.

59. A method as claimed in claim 55, wherein the heterologous antigen is a human pathogen-specific antigen.

60. A method as claimed in claim 59, wherein the human pathogen-specific antigen is a viral antigen.

61. A method as claimed in claim 60, in which the viral antigen is selected from the group consisting of: human immuno-deficiency virus, simian immuno-deficiency virus, Kaposi's sarcoma-associated herpesvirus, Herpes simplex virus 1, Herpes simplex virus 2, Epstein Barr virus, hepatitis B virus, human papillomavirus, influenza virus, monkeypox virus, West Nile virus, Chikungunya virus, Ebola virus, hepatitis C virus, poliovirus, dengue virus, herpes virus B, Marburg virus, SARS virus, and MERS virus.

62. A method as claimed in claim 60, wherein the viral antigen is a viral protein, an epitope or antigenic fragment thereof.

63. A method as claimed in claim 55, wherein the heterologous antigen is a bacterial antigen.

64. A method as claimed in claim 63, wherein the bacterial antigen is a bacterial protein, an epitope or antigenic fragment thereof.

65. A method of preparing a recombinant herpesvirus-based vector comprising the steps of:
providing a nucleic acid sequence encoding a heterologous antigen;
selecting a promoter for controlling the expression of the antigen,
in which the promoter is selected to express at a time selected to provide a required immune response in a subject selected from the group consisting of: T-cell biased; antibody biased; balanced T-cell and antibody.
